(19) United States
(12) Patent Application Publication
(10) Pub. No.: US 2024/0366099 A1
(43) Pub. Date: Nov. 7, 2024

(54) BLOOD PRESSURE MEASUREMENT METHOD, APPARATUS, DEVICE AND READABLE STORAGE MEDIUM

(71) Applicant: Shenzhen AOJ Medical Technology Co., Ltd., Shenzhen (CN)

(72) Inventor: Yongjie Gao, Shenzhen (CN)

(21) Appl. No.: 18/775,373

(22) Filed: Jul. 17, 2024

Related U.S. Application Data
(63) Continuation of application No. PCT/CN2022/098749, filed on Jun. 14, 2022.

(30) Foreign Application Priority Data
Jan. 25, 2022 (CN) 202210085229.4

(51) Int. Cl. A61B 5/0225; A61B 5/021

(52) U.S. Cl. CPC A61B 5/0225; A61B 5/02141

(57) ABSTRACT

The present application provides a blood pressure measurement method, apparatus, device, and readable storage medium. The method includes: controlling a cuff air bladder to depressurize in a state of pressurization completion; collecting a heart sound signal sequence and a cuff pressure time sequence in real-time; calculating a peak heart sound point time sequence based on the heart sound signal sequence; and finding a cuff pressure value at a corresponding moment in the cuff pressure time sequence as a blood pressure measurement value based on a moment of occurrence of a target peak heart sound point in the peak heart sound point time sequence. In the present application, the standardization of listening to the Korotkoff sounds is carried out, and the collected signals are automatically processed and recognized to obtain the blood pressure measurement value, which effectively improves the convenience and accuracy of the blood pressure measurement.

### CROSS-REFERENCE TO RELATED APPLICATIONS

The present application is a continuation of International Application No. PCT/CN2022/098749, filed on Jun. 14, 2022, which claims priority to Chinese patent application No. 202210085229.4, filed on Jan. 25, 2022. The entire contents of these applications are incorporated herein by reference in their entirety.

### TECHNICAL FIELD

The present application relates to the technical field of medical electronic devices, in particular to a blood pressure measurement method, apparatus, device, and readable storage medium.

### BACKGROUND

Blood pressure is the pressure exerted by the flow of blood against the walls of blood vessels as blood circulates through the body. It is one of the most important vital signs, serving as a critical indicator for disease diagnosis, treatment efficacy evaluation, and overall health assessment. Hypertension is one of the most definitive and significant risk factors for cardiovascular and cerebrovascular diseases. In China, over 300 million people suffer from hypertension, making blood pressure monitoring a crucial parameter in both clinical diagnosis and home health management. Additionally, with the global aging population and rising incidence of chronic diseases, the accuracy, accessibility, and home use of blood pressure monitoring devices have become essential needs.

Currently, the medical industry widely adopts the Korotkoff sound method as the “gold standard” for blood pressure measurement. The measurement process involves using a cuff to occlude arterial blood flow and then slowly releasing the pressure while listening to the Korotkoff sounds with a stethoscope. Initially, strong and clear Korotkoff sounds can be heard, which gradually become fainter until they disappear. The first Korotkoff sound heard indicates the systolic pressure, as read from the mercury sphygmomanometer. As the cuff continues to deflate, the Korotkoff sounds gradually diminish until they disappear; the pressure at which the last Korotkoff sound is heard corresponds to the diastolic pressure.

However, the Korotkoff sound method for measuring blood pressure has some drawbacks: First, it requires the measurer to have the professional knowledge to discern the pressure changes indicated by the different phases of Korotkoff sounds, necessitating specialized medical training. Second, it demands precise coordination of the measurer's eyes, hands, and ears to accurately read the pressure gauge when the Korotkoff sounds appear or disappear. Third, the Korotkoff method cannot be used independently by the subject, and it requires a professional to perform the measurement. Fourth, individual variations in the measurer's visual and auditory reaction speed and sensitivity can lead to significant errors, and changes in the measurer's age and physiological conditions, such as hearing ability, can also affect their response to the Korotkoff sounds and the accuracy of the readings.

### SUMMARY

Embodiments of the present application provide a blood pressure measurement method, apparatus, device, and readable storage medium, which can at least solve the problems of low measurement convenience and low measurement accuracy of the blood pressure measurement methods provided in the related technology.

A first aspect of the embodiments of the present application provides a blood pressure measurement method, applied to an electronic blood pressure measurement device, comprising:
controlling a cuff air bladder to depressurize when the cuff air bladder is in a state of pressurization completion;
collecting a heart sound signal sequence as well as a cuff pressure time sequence in real-time by a heart sound collecting unit during a depressurization process;
calculating a peak heart sound point time sequence based on the heart sound signal sequence;
and
finding a cuff pressure value at a corresponding moment in the cuff pressure time sequence as a blood pressure measurement value based on a moment of occurrence of a target peak heart sound point in the peak heart sound point time sequence.

A second aspect of embodiments of the present application provides a blood pressure measurement apparatus, applied to an electronic blood pressure measurement device, comprising:
a control module configured to control a cuff air bladder to depressurize when the cuff air bladder is in a state of pressurization completion;
a collecting module configured to collect a heart sound signal sequence as well as a cuff pressure time sequence in real-time by a heart sound collecting unit during a depressurization process;
a calculation module configured to calculate a peak heart sound point time sequence based on the heart sound signal sequence; and
a finding module configured to find a cuff pressure value at a corresponding moment in the cuff pressure time sequence as a blood pressure measurement value based on a moment of occurrence of a target peak heart sound point in the peak heart sound point time sequence.

A third aspect of embodiments of the present application provides an electronic blood pressure measurement device comprising: a memory and a processor, wherein the processor is configured to execute a computer program stored on the memory, and the processor, when executing the computer program, realizes each step in the above-described blood pressure measurement method provided in the first aspect of embodiments of the present application.

A fourth aspect of the embodiment of the present application provides a computer-readable storage medium having a computer program stored thereon, wherein the computer program, when executed by the processor, realizes the respective steps in the blood pressure measurement method provided in the above-described first aspect of the embodiment of the present application.

As can be seen from the above, according to the blood pressure measurement method, apparatus, device, and readable storage medium provided in the embodiment of the present application, the cuff air bladder is controlled to depressurize when the cuff air bladder is in a state of pressurization completion; a heart sound signal sequence as well as a cuff pressure time sequence are collected in real-time by a heart sound collecting unit during a depressurization process; a peak heart sound point time sequence is calculated based on the heart sound signal sequence; and a cuff pressure value at a corresponding moment in the cuff pressure time sequence is found as a blood pressure measurement value based on a moment of occurrence of a target peak heart sound point in the peak heart sound point time sequence. Through the implementation of the present application, the standardization of listening to the Korotkoff sounds is carried out based on the digital method, and the collected signals are automatically processed and recognized to obtain the blood pressure measurement value, which effectively improves the convenience and accuracy of the blood pressure measurement.

### BRIEF DESCRIPTION OF THE DRAWINGS

FIG. 1 shows a basic flowchart of a blood pressure measurement method provided in a first embodiment of the present application.

FIG. 2 shows a schematic diagram of a conventional pressurization scheme provided by the first embodiment of the present application.

FIG. 3 shows a schematic diagram of an improved pressurization scheme provided by the first embodiment of the present application.

FIG. 4 shows a schematic diagram of a peak detection provided by the first embodiment of the present application.

FIG. 5 shows a refined flowchart of a blood pressure measurement method provided in a second embodiment of the present application.

FIG. 6 shows a schematic diagram of program modules of a blood pressure measurement apparatus provided in a third embodiment of the present application.

FIG. 7 shows a structural schematic diagram of an electronic blood pressure measurement device provided in a fourth embodiment of the present application.

### DETAILED DESCRIPTION OF THE EMBODIMENTS

In order to make the inventive object, features, and advantages of the present application more obvious and understandable, the technical solutions in the embodiments of the present application will be described clearly and completely in the following in conjunction with the accompanying drawings in the embodiments of the present application. Obviously, the described embodiments are only a part of the present application rather than all of the embodiments. Based on the embodiments in the present application, all other embodiments obtained by persons skilled in the art without making creative labor are within the scope of protection of the present application.

In the description of the embodiments of the present application, it should be understood that the terms “length”, “width”, “up”, “down”, “front”, “back”, “left”, “right”, “vertical”, “horizontal”, “top”, “bottom”, “inside”, “outside” and the like indicate orientations or positional relationships based on those shown in the accompanying drawings, and are only intended to facilitate the description of the embodiments of the present application and to simplify the description, and are not intended to indicate or imply that the device or element referred to must have a particular orientation, be constructed and operated with a particular orientation, and therefore are not to be construed as a limitation of the present application.

Furthermore, the terms “first” and “second” are used for descriptive purposes only and are not to be understood as indicating or implying relative importance or implicitly specifying the number of technical features indicated. Thus, a feature defined with “first”, “second” may expressly or implicitly include one or more such features. In the description of the embodiments of the present application, “more than one” means two or more, unless otherwise expressly and specifically limited.

In embodiments of the present application, unless otherwise expressly specified and limited, the terms “mount”, “joint”, “connect”, “fixing”, and the like should be understood broadly. For example, it may be a fixed connection, a removable connection, or a one-piece connection; it may be a mechanical connection or an electrical connection; it may be a direct connection or an indirect connection through an intermediate medium, and it may be a connection within the two elements or an interactive relationship between the two elements. For those of ordinary skill in the art, the specific meanings of the above terms in the embodiments of the present application may be understood on a case-by-case basis.

Described below are only preferred embodiments of the present application, and are not intended to limit the present application, and any modifications, equivalent substitutions, improvements, etc. made within the spirit and principles of the present application shall be included in the scope of protection of the present application.

In order to solve the problem of low measurement convenience and measurement accuracy of the blood pressure measurement method provided in the related art, a first embodiment of the present application provides a blood pressure measurement method applied to an electronic blood pressure measurement device. The electronic blood pressure measurement device may include a cuff, a heart sound collecting unit, an air pressure sensor, a processing unit, a pressure control unit, a human-computer interaction unit. A cuff air bladder is provided in the cuff. The heart sound collecting unit may be a piezoelectric film-based sound vibration digitizing sensor, a sound sensing MEMS chip, a microphone, etc. During the process of inflating/deflating the cuff air bladder, the heart sound collecting unit is placed on the upper side of the brachial artery in an independent or integrated manner, to collect the Korotkoff sound of the brachial artery during the process of the flow of the brachial artery from being blocked to being opened. The processing unit runs the Korotkoff sound detection algorithm to process the collected signal and accurately obtain the beginning and end of the Korotkoff sound to determine the systolic and diastolic blood pressure. The human-computer interaction unit may include a loudspeaker for playing the Korotkoff sound of the brachial artery outwardly, so that the tester or the subject can clearly hear the appearance and disappearance of the Korotkoff sound and can visually compare and contrast with the actual measurement of the displayed blood pressure value to make a judgment.

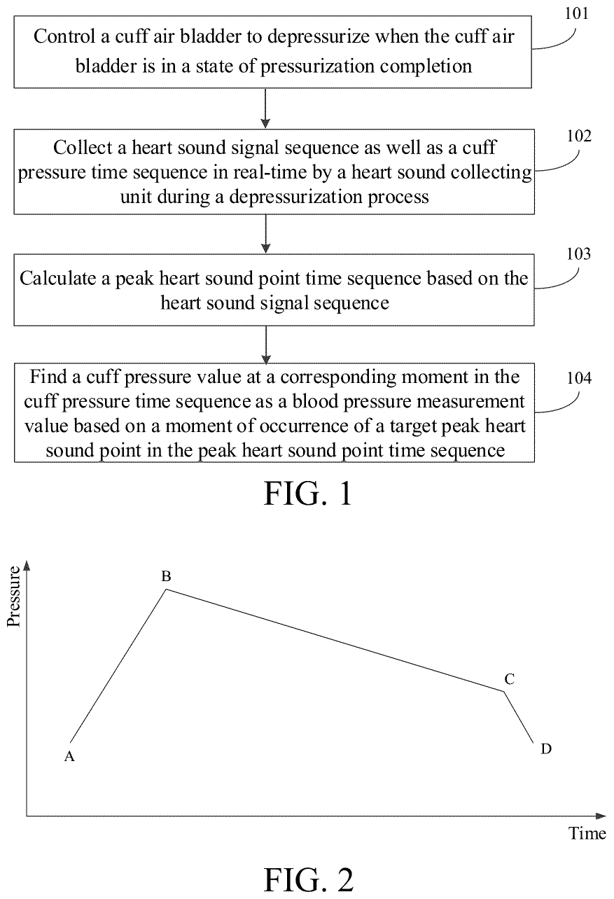

FIG. 1 is a basic schematic flowchart of a blood pressure measurement method provided in this embodiment, and the blood pressure measurement method includes the following steps.

Step 101, a cuff air bladder is controlled to depressurize when the cuff air bladder is in a state of pressurization completion.

Specifically, when the electronic blood pressure measurement device is in a blood pressure measurement working mode after startup, the air pump is first controlled by the processing unit to pressurize and inflate the cuff air bladder to reach a target pressure value, and then the cuff air bladder is triggered to depressurize and enter the blood pressure measurement state.

In an embodiment of this embodiment, the step of controlling the cuff air bladder to depressurize includes: controlling the cuff air bladder to depressurize at a first gas flow rate during a first depressurization phase; controlling the cuff air bladder to depressurize at a second gas flow rate during a second depressurization phase when a first heart sound signal is captured by the heart sound collecting unit; controlling the cuff air bladder to depressurize at a third gas flow rate during a third depressurization phase when the heart sound collecting unit a last heart sound signal is collected. The first gas flow rate is greater than the second gas flow rate, and the third gas flow rate is greater than the first gas flow rate.

FIG. 2 is a schematic diagram of a conventional pressurization scheme provided by this embodiment. The phase A to B is the cuff pressurization phase, during which the air bladder is rapidly pressurized. When the maximum pressure is reached at point B, deflation and depressurization is begun and signal collection is begun at the same time, and the phase B to C is the slow depressurization phase. When the diastolic pressure is detected at point C, the rapid depressurization is carried out in the phase C to D.

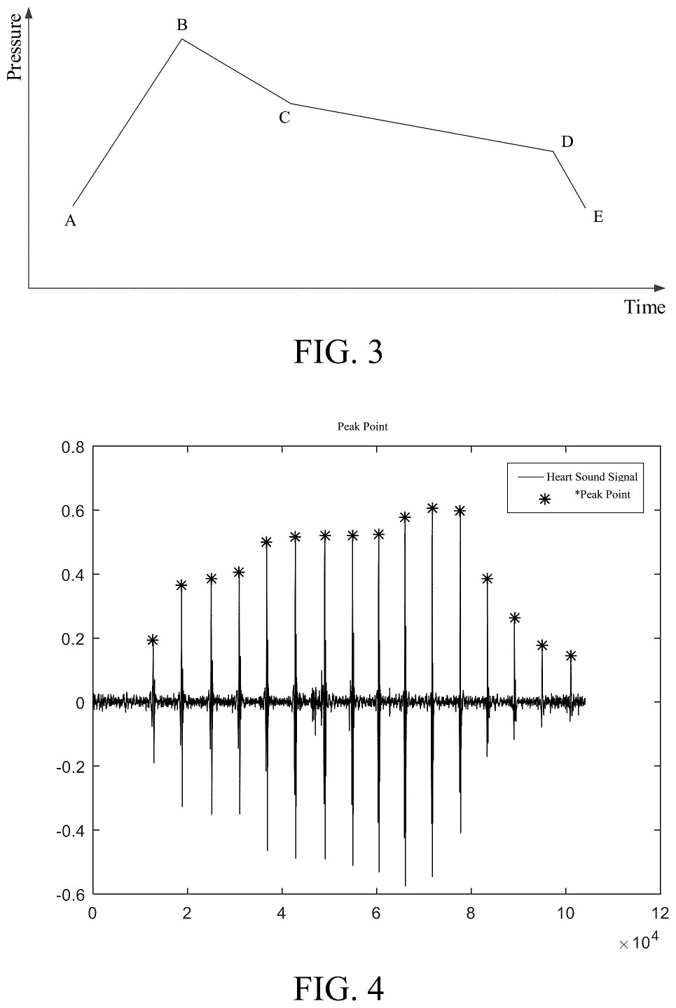

FIG. 3 is a schematic diagram of an improved pressurization solution provided by this embodiment. The phase A to B is the cuff pressurization phase, which is the same as the solution of FIG. 2, and the air bladder is rapidly pressurized during this phase. The difference is that the rapid depressurization is started in the first depressurization phase, i.e., the phase B to C in this embodiment. When a first Korotkoff sound is detected at point C, the depressurization is slowed down in a second depressurization phase, i.e., the phase C to D, in order to have a higher quality of the signal acquired in this phase, and the pressure change per heartbeat interval becomes smaller when the depressurization rate is slowed down, resulting in higher measurement accuracy. Then, when the last Korotkoff sound is detected at point D, the rapid depressurization is carried out at the maximum depressurization rate in the third depressurization phase, i.e., the phase D to E.

It should be noted that in one implementation of this embodiment, a pneumatic pressure control unit for controlling the pressure adjustment of the cuff air bladder may include a proportional valve, and the proportional valve is completely closed during the pressurization process without leakage nozzles, which provides a faster pressurization speed relative to the conventional solution. In the depressurization process, the degree of opening of the proportional valve may be adjusted to achieve depressurization phases with different gas flow rates. Of course, in another implementation of this embodiment, the pneumatic pressure control unit may include a solenoid valve and a plurality of leak-off valves with different gas flow rates, and in the depressurization process, the depressurization may be realized by controlling the leakage of gas from different leak-off valves through the solenoid valve in different depressurization phases.

Further, in an implementation of this embodiment, the step of controlling the cuff air bladder to depressurize at the second gas flow rate during the second depressurization phase includes: dividing the second depressurization phase into a plurality of sub-depressurization phases and controlling the cuff air bladder to depressurize at a corresponding second gas flow rate selected from a set of preset second gas flow rate values in the plurality of the sub-depressurization phases.

Specifically, in practical application, in order to further enhance the measurement sensitivity and the measurement progress, a plurality of sub-depressurization phases may be further set up in the above-mentioned second depressurization phase, and then different gas flow rates are set up for different sub-depressurization phases to control depressurization of the cuff air bladder.

Step 102: a heart sound signal sequence as well as a cuff pressure time sequence are collected in real-time by a heart sound collecting unit during a depressurization process.

Specifically, the heart sound signal and the time sequence of the cuff pressure value are collected synchronously during the depressurization of the cuff air bladder in this embodiment. In practical application, in order to improve the signal quality, the heart sound signal may be band-pass filtered at 20-200 Hz to reduce the noise interference, and the resulting sequence of the heart sound signal is x, and the sequence of the cuff pressure change over time is p.

Step 103: a peak heart sound point time sequence is calculated based on the heart sound signal sequence.

In an implementation of this embodiment, the step of calculating the peak heart sound point time sequence based on the heart sound signal sequence includes: intercepting a first heart sound signal sequence N seconds before the heart sound signal sequence, and sorting the first heart sound signal sequence in ascending order to obtain a second heart sound signal sequence; calculating an average value of M heart sound signals after the second heart sound signal sequence to obtain a noise amplitude threshold; obtaining valid heart sound signals from the heart sound signal sequence based on the noise amplitude threshold to be deposited into a heart sound vector, and recording a time vector corresponding to the heart sound vector; and calculating the peak heart sound point time sequence based on the heart sound vector and the time vector.

Specifically, in this embodiment, at the initial detection phase of the electronic blood pressure measurement device, the cuff pressure is too high, and usually there is no heart sound signal, so that the sound detection signal in the first N seconds (e.g., 0.01 to 1 second) may be taken as the ambient noise. Therefore, the n-point heart sound data sequence X start before the N seconds is selected in this embodiment. In the practical application, the noise is random, and the positive and negative values appear randomly, so that averaging the heart sound data directly may be close to zero. Thus, in order to avoid the noise threshold estimation of a small value in this embodiment, which affects the subsequent peak detection, the foregoing heart sound data sequence xₛₜₐᵣₜ is sorted in ascending order to obtain yₛₜₐᵣₜ. After sorting the noise sequence in ascending order, the larger values are all concentrated at the end of the ascending sequence yₛₜₐᵣₜ, and the ascending sequence is expressed as follows: yₛₜₐᵣₜ=Sort (xₛₜₐᵣₜ).

Next, the M (e.g., n/4) heart sound signals at the tail of the heart sound signal sequence yₛₜₐᵣₜ after the ascending order sorting are averaged as the noise amplitude threshold thr, which is expressed by the following formula:

$$thr = {\sum }_{i = 3 n / 4}^{n} {y}_{start}^{i} / n / 4.$$

In this embodiment, considering that the heart sound signals are generally greater than the noise signals by a factor of 5 or more, whereby 5*thr may be used as the signal comparison threshold. The heart sound signals in the heart sound signal sequence x are compared with this signal comparison threshold, and then the heart sound signals greater than 5*thr are deposited into the heart sound vector xₚₑₐₖₛ, the heart sound signals less than thr are set to 0, and the time vector fₚₑₐₖₛ Of xₚₑₐₖₛ is retained.

Further, in an implementation of this embodiment, the step of calculating the peak heart sound point time sequence based on the heart sound vector and the time vector includes: performing a difference operation on all the valid heart sound signals in the heart sound vector to obtain a difference sequence; performing a peak detection on the difference sequence and the time vector based on a predetermined peak detection formula to obtain a peak point sequence and a corresponding time sequence; and determining the peak heart sound point time sequence based on the peak point sequence and the corresponding time sequence.

Specifically, in this embodiment, the difference operation is performed on the xₚₑₐₖₛ to obtain a difference sequence of diffₚₑₐₖₛ. The difference calculation formula is expressed as:

$${diff}_{peaks} = {x}_{peaks}^{i + 1} - {x}_{peaks}^{i} , ( i = 0 , 1 , 2 ... . n - 1 ) .$$

FIG. 4 is a schematic diagram showing a peak detection provided in this embodiment. In this embodiment, the peak detection is performed on the difference sequence diffₚₑₐₖₛ, and the peak detection principle is that the derivatives at both ends of the maximum value of a segment of a curve are dissimilar, and the derivatives on both sides of the maximum value, i.e., the point at which the product of the two differential values is negative is the point at which the peak point is located. Based on the above principle, the peak point sequence hₚₑₛₖₛ and its corresponding time sequence hₜᵢₘₑ meeting the peak detection can be found in accordance with the difference sequence derived from the preceding steps, and the moments without peaks are not recorded. The peak detection formula is as follows:

$${h}_{peaks} = \{ \begin{matrix}{diff}_{peaks}^{i} * {diff}_{peaks}^{i + 1} < 0 & {h}_{time}^{j} = {t}_{peaks}^{i} \\ other & {h}_{time}^{j} = [ ]\end{matrix} .$$

Step 104, a cuff pressure value at a corresponding moment in the cuff pressure time sequence is found as a blood pressure measurement value based on a moment of occurrence of a target peak heart sound point in the peak heart sound point time sequence.

Specifically, in this embodiment, the cuff pressure value at the corresponding moment in the cuff pressure time sequence is found as a high-pressure measurement value of the blood pressure based on the moment of occurrence of a first peak heart sound point in the peak heart sound point time sequence, and the cuff pressure value of the corresponding moment in the cuff pressure time sequence is found as a low-pressure measurement value of the blood pressure based on the moment of occurrence of a last peak heart sound point in the peak heart sound point time sequence.

The cuff pressure corresponding to the first time point of the peak point time sequence hₜᵢₘₑ is the high-pressure measurement value P_{high} of the blood pressure, and the cuff pressure corresponding to the last time point is the low-pressure measurement value Plow of the blood pressure. The correspondence is as follows:

$${p}_{high} = p ( {h}_{time} ( 1 ) ) , {p}_{low} = p ( {h}_{time} ( end ) ) .$$

Of course, it should be understood that this embodiment is not limited to taking the high-pressure measurement value and the low-pressure measurement value as the final output blood pressure value. In practical application, the cuff pressure corresponding to other peak points may also be selected as the specific blood pressure measurement value for output.

In addition, it should be noted that in this embodiment, the pressure value and the volume map of the Korotkoff sound signal can be displayed in real-time on the display screen during the entire measurement process, and the simultaneous output of Korotkoff sound through the speaker is supported. Besides, after the measurement is finished, the measurement results of systolic pressure and diastolic pressure are displayed by the display screen, and the measurement results are voice broadcast by the speaker.

Based on the above technical solution of the embodiment of the present application, the cuff air bladder is controlled to depressurize when the cuff air bladder is in a state of pressurization completion; a heart sound signal sequence as well as a cuff pressure time sequence are collected in real-time by a heart sound collecting unit during a depressurization process; a peak heart sound point time sequence is calculated based on the heart sound signal sequence; and a cuff pressure value at a corresponding moment in the cuff pressure time sequence is found as a blood pressure measurement value based on a moment of occurrence of a target peak heart sound point in the peak heart sound point time sequence. Through the implementation of the present application, the standardization of listening to the Korotkoff sounds is carried out based on the digital method, and the collected signals are automatically processed and recognized to obtain the blood pressure measurement value, which effectively improves the convenience and accuracy of the blood pressure measurement.

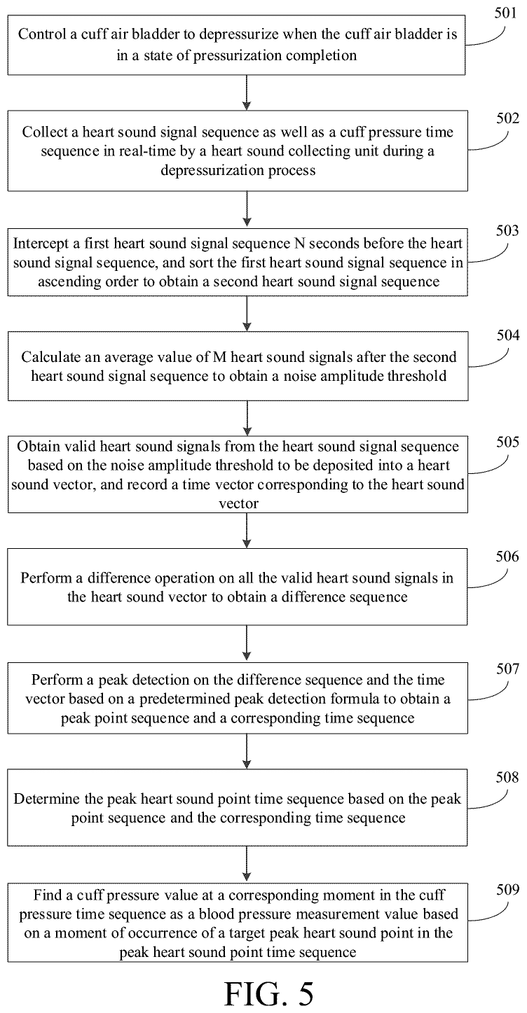

The method in FIG. 5 is a refined blood pressure measurement method provided in a second embodiment of the present application, and the blood pressure measurement method includes the following steps.

Step 501, a cuff air bladder is controlled to depressurize when the cuff air bladder is in a state of pressurization completion.

Step 502, a heart sound signal sequence as well as a cuff pressure time sequence are collected in real-time by a heart sound collecting unit during a depressurization process.

Step 503: a first heart sound signal sequence N seconds before the heart sound signal sequence is intercepted, and the first heart sound signal sequence is sorted in ascending order to obtain a second heart sound signal sequence.

Step 504: an average value of M heart sound signals after the second heart sound signal sequence is calculated to obtain a noise amplitude threshold.

Step 505: valid heart sound signals are obtained from the heart sound signal sequence based on the noise amplitude threshold to be deposited into a heart sound vector, and a time vector corresponding to the heart sound vector is recorded.

Step 506: a difference operation is performed on all the valid heart sound signals in the heart sound vector to obtain a difference sequence.

Step 507: a peak detection is performed on the difference sequence and the time vector based on a predetermined peak detection formula to obtain a peak point sequence and a corresponding time sequence.

Step 508: the peak heart sound point time sequence is determined based on the peak point sequence and the corresponding time sequence.

Step 509: a cuff pressure value at a corresponding moment in the cuff pressure time sequence is found as a blood pressure measurement value based on a moment of occurrence of a target peak heart sound point in the peak heart sound point time sequence.

It should be understood that the magnitude of the serial numbers of the steps in this embodiment does not imply the sequence of the order of execution of the steps, and the order of execution of the steps should be determined by their functions and inherent logic without constituting a sole limitation of the process of implementing the embodiments of the present application.

It should be noted that in this embodiment, the blood pressure measurement is based on the Korotkoff sound method principle, but the process of listening to Korotkoff sounds is standardized through digital means. This eliminates the need for the measurer to possess professional measurement knowledge to distinguish the pressure changes represented by Korotkoff sounds at different phases. Additionally, the measurer does not need precise coordination of eyes, hands, and ears to automatically and accurately identify pressure values when Korotkoff sounds appear or disappear. Furthermore, there are no special requirements for the measurer's reaction speed and sensitivity of eyes and ears, and changes in the measurer's age and physiological conditions, such as hearing, do not affect the response to Korotkoff sound changes and readings. Moreover, this method does not rely on the oscillometric method for measurement, it precisely measures the actual blood flow and blood pressure of the individual, solving the problems of inaccuracy and lack of individual-specific measurements associated with the oscillometric method.

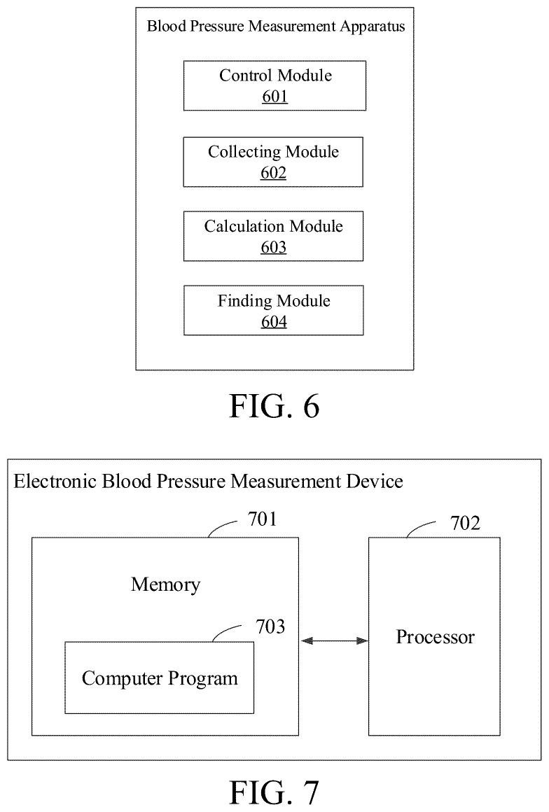

FIG. 6 shows a blood pressure measurement apparatus provided in a third embodiment of the present application. The blood pressure measurement apparatus may be used to realize the blood pressure measuring method in the preceding embodiment. As shown in FIG. 6, the blood pressure measurement apparatus mainly includes the following modules.

A control module 601 is configured to control a cuff air bladder to depressurize when the cuff air bladder is in a state of pressurization completion.

A collecting module 602 is configured to collect a heart sound signal sequence as well as a cuff pressure time sequence in real-time by a heart sound collecting unit during a depressurization process.

A calculation module 603 is configured to calculate a peak heart sound point time sequence based on the heart sound signal sequence.

A finding module 604 is configured to find a cuff pressure value at a corresponding moment in the cuff pressure time sequence as a blood pressure measurement value based on a moment of occurrence of a target peak heart sound point in the peak heart sound point time sequence.

In some implementations of this embodiment, the calculation module is specifically configured to: intercept a first heart sound signal sequence N seconds before the heart sound signal sequence, and sort the first heart sound signal sequence in ascending order to obtain a second heart sound signal sequence; calculate an average value of M heart sound signals after the second heart sound signal sequence to obtain a noise amplitude threshold; obtain valid heart sound signals from the heart sound signal sequence based on the noise amplitude threshold to be deposited into a heart sound vector, and record a time vector corresponding to the heart sound vector; calculate the peak heart sound point time sequence based on the heart sound vector and the time vector.

Further, in some implementations of this embodiment, the calculation module, in performing the above function of calculating the peak heart sound point time sequence based on the heart sound vector and the time vector, is specifically configured to: perform a difference operation on all the valid heart sound signals in the heart sound vector to obtain a difference sequence; perform a peak detection on the difference sequence and the time vector based on a predetermined peak detection formula to obtain a peak point sequence and a corresponding time sequence; and determine the peak heart sound point time sequence based on the peak point sequence and the corresponding time sequence

Further, in some implementations of this embodiment, the peak detection formula is expressed as:

$${h}_{peaks} = \{ \begin{matrix}{diff}_{peaks}^{i} * {diff}_{peaks}^{i + 1} < 0 & {h}_{time}^{j} = {t}_{peaks}^{i} \\ other & {h}_{time}^{j} = [ ]\end{matrix} .$$

In some implements of this embodiment, the control module is specifically configured to: control the cuff air bladder to depressurize at a first gas flow rate during a first depressurization phase; control the cuff air bladder to depressurize at a second gas flow rate during a second depressurization phase when a first heart sound signal is captured by the heart sound collecting unit; and control the cuff air bladder to depressurize at a third gas flow rate during a third depressurization phase when the heart sound collecting unit a last heart sound signal is collected. The first gas flow rate is greater than the second gas flow rate, and the third gas flow rate is greater than the first gas flow rate.

Further, in some implements of this embodiment, the control module, in performing the above-described function of controlling the cuff air bladder to depressurize at the second gas flow rate during the second depressurization phase, is specifically configured to: divide the second depressurization phase into a plurality of sub-depressurization phases and control the cuff air bladder to depressurize at a corresponding second gas flow rate selected from a set of preset second gas flow rate values in the plurality of the sub-depressurization phases

In some implements of this embodiment, the finding module is specifically configured to: find the cuff pressure value at the corresponding moment in the cuff pressure time sequence as a high-pressure measurement value of the blood pressure based on the moment of occurrence of a first peak heart sound point in the peak heart sound point time sequence, and find the cuff pressure value of the corresponding moment in the cuff pressure time sequence as a low-pressure measurement value of the blood pressure based on the moment of occurrence of a last peak heart sound point in the peak heart sound point time sequence.

It should be noted that the blood pressure measurement methods in the first and second embodiments may all be realized based on the blood pressure measurement apparatus provided in this embodiment. It can be clearly understood by a person of ordinary skill in the field that, for the convenience and conciseness of the description, the specific working process of the blood pressure measurement apparatus described in this embodiment may be referred to the corresponding process in the foregoing embodiment of the method, and will not be repeated herein.

According to the blood pressure measurement apparatus provided in this embodiment, the cuff air bladder is controlled to depressurize when the cuff air bladder is in a state of pressurization completion; a heart sound signal sequence as well as a cuff pressure time sequence are collected in real-time by a heart sound collecting unit during a depressurization process; a peak heart sound point time sequence is calculated based on the heart sound signal sequence; and a cuff pressure value at a corresponding moment in the cuff pressure time sequence is found as a blood pressure measurement value based on a moment of occurrence of a target peak heart sound point in the peak heart sound point time sequence. Through the implementation of the present application, the standardization of listening to the Korotkoff sounds is carried out based on the digital method, and the collected signals are automatically processed and recognized to obtain the blood pressure measurement value, which effectively improves the convenience and accuracy of the blood pressure measurement.

FIG. 7 shows an electronic blood pressure measurement device provided in a fourth embodiment of the present application. The electronic blood pressure measurement device can be used to realize the blood pressure measurement method in the preceding embodiment, and mainly includes:
a memory 701, a processor 702, and a computer program 703 stored on the memory 701 and runnable on the processor 702. The memory 701 and the processor 702 are connected by communication. The processor 702 implements the method in the first or second embodiment when it executes the computer program 703. The number of processors may be one or more.

The memory 701 may be a high-speed random access memory (RAM), or a non-volatile memory, such as a disk memory. The memory 701 is configured to store executable program code, and the processor 702 is coupled to the memory 701.

Further, embodiments of the present application also provide a computer-readable storage medium which may be provided in an electronic device in the embodiments described above and may be the memory in the embodiment shown in FIG. 7.

The computer-readable storage medium has stored a computer program, and the computer program implements the blood pressure measurement method in the foregoing embodiments when executed by a processor. Further, the computer-readable storage medium may also be a USB flash disk, a removable hard disk, a read-only memory (ROM), a RAM, a magnetic disk, a CD-ROM, or various other media that can store the program code.

In the several embodiments provided in the present application, it should be understood that the devices and methods disclosed, may be realized in other ways. For example, the above-described embodiments of the device are merely schematic, e.g., the division of modules, which is merely a logical functional division, may be divided in other ways when actually implemented, e.g., multiple modules or components may be combined or may be integrated into another system, or some features may be ignored, or not implemented. At another point, the mutual coupling, direct coupling or communication connection shown or discussed may be an indirect coupling or communication connection through some interface, device, or module, which may be electrical, mechanical, or otherwise.

The modules illustrated as separate components may or may not be physically separate, and the components shown as modules may or may not be physical modules, i.e., they may be located in a single place or they may be distributed to a plurality of network modules. Some or all of these modules may be selected to fulfill the purpose of the embodiment scheme according to practical needs.

In addition, the various functional modules in the various embodiments of the present application may be integrated in a single processing module, or the individual modules may be physically present separately, or two or more modules may be integrated in a single module. The above integrated modules may be implemented either in the form of hardware or in the form of software function modules.

The integrated modules, when implemented in the form of software function modules and sold or used as separate products, may be stored in a computer-readable storage medium. Based on such understanding, the technical solution of the present application may be embodied, in essence or as a contribution to the prior art, or in whole or in part, in the form of a software product, which is stored in a computer-readable storage medium and includes a number of instructions to enable a computer device (which may be a personal computer, a server, or a network device, etc.) to perform all or part of the steps of the method of the various embodiments of the present application. The above-mentioned readable storage medium includes a USB flash disk, a removable hard disk, a ROM, a RAM, a diskette, a CD-ROM, or other media that can store program code.

It is to be noted that the above-mentioned method embodiments are expressed as a series of action combinations for the sake of simplicity of description, but the person skilled in the art should be aware that the present application is not limited by the order of the described actions, because some of the steps may be carried out in a different order or at the same time according to the present application. Secondly, the person skilled in the art should also be aware that the embodiments described in the specification are preferred embodiments, and the actions and modules involved are not necessarily necessary for the present application.

In the above embodiments, the description of each embodiment has its own focus, and the part that is not described in detail in a certain embodiment can be referred to as the relevant description of other embodiments.

Described above are the blood pressure measurement method, apparatus, device, and readable storage medium provided in the present application. For the technical personnel in the field, based on the ideas of the embodiments of the present application, there may be changes in the specific implementation and application scope. In summary, the contents of this specification should not be construed as a limitation of the present application.

What is claimed is:

1. A blood pressure measurement method, applied to an electronic blood pressure measurement device, comprising:
controlling a cuff air bladder to depressurize when the cuff air bladder is in a state of pressurization completion;
collecting a heart sound signal sequence as well as a cuff pressure time sequence in real time by a heart sound collecting unit during a depressurization process;
calculating a peak heart sound point time sequence based on the heart sound signal sequence; and
finding a cuff pressure value at a corresponding moment in the cuff pressure time sequence as a blood pressure measurement value based on a moment of occurrence of a target peak heart sound point in the peak heart sound point time sequence.

2. The blood pressure measurement method of claim 1, wherein the step of calculating the peak heart sound point time sequence based on the heart sound signal sequence comprises:
intercepting a first heart sound signal sequence N seconds before the heart sound signal sequence, and sorting the first heart sound signal sequence in ascending order to obtain a second heart sound signal sequence;
calculating an average value of M heart sound signals after the second heart sound signal sequence to obtain a noise amplitude threshold;
obtaining valid heart sound signals from the heart sound signal sequence based on the noise amplitude threshold to be deposited into a heart sound vector, and recording a time vector corresponding to the heart sound vector; and
calculating the peak heart sound point time sequence based on the heart sound vector and the time vector.

3. The blood pressure measurement method of claim 2, wherein the step of calculating the peak heart sound point time sequence based on the heart sound vector and the time vector comprises:
performing a difference operation on all the valid heart sound signals in the heart sound vector to obtain a difference sequence;
performing a peak detection on the difference sequence and the time vector based on a predetermined peak detection formula to obtain a peak point sequence and a corresponding time sequence; and
determining the peak heart sound point time sequence based on the peak point sequence and the corresponding time sequence.

4. The blood pressure measurement method of claim 3, wherein the peak detection formula is expressed as:
$${h}_{peaks} = \{ \begin{matrix}{diff}_{peaks}^{i} * {diff}_{peaks}^{i + 1} < 0 & {h}_{time}^{j} = {t}_{peaks}^{i} \\ other & {h}_{time}^{j} = [ ]\end{matrix} ;$$
wherein hₚₑₐₖₛ denotes the peak point sequence, hₜᵢₘₑ denotes the time sequence, diffₚₑₐₖₛ denotes the difference sequence, and tₚₑₐₖₛ denotes the time vector.

5. The blood pressure measurement method of claim 1, wherein the step of controlling the cuff air bladder to depressurize comprises:
controlling the cuff air bladder to depressurize at a first gas flow rate during a first depressurization phase;
controlling the cuff air bladder to depressurize at a second gas flow rate during a second depressurization phase when a first heart sound signal is captured by the heart sound collecting unit;
controlling the cuff air bladder to depressurize at a third gas flow rate during a third depressurization phase when the heart sound collecting unit a last heart sound signal is collected;
wherein the first gas flow rate is greater than the second gas flow rate, and the third gas flow rate is greater than the first gas flow rate.

6. The blood pressure measurement method of claim 5, wherein the step of controlling the cuff air bladder to depressurize at the second gas flow rate during the second depressurization phase comprises:
dividing the second depressurization phase into a plurality of sub-depressurization phases and controlling the cuff air bladder to depressurize at a corresponding second gas flow rate selected from a set of preset second gas flow rate values in the plurality of the sub-depressurization phases.

7. The blood pressure measurement method of claim 1, wherein the step of finding the cuff pressure value at the corresponding moment in the cuff pressure time sequence as the blood pressure measurement value based on the moment of occurrence of the target peak heart sound point in the peak heart sound point time sequence comprises:
finding a cuff pressure value at a corresponding moment in the cuff pressure time sequence as a high-pressure measurement value of the blood pressure based on a moment of occurrence of a first peak heart sound point in the peak heart sound point time sequence, and finding a cuff pressure value of a corresponding moment in the cuff pressure time sequence as a low-pressure measurement value of the blood pressure based on a moment of occurrence of a last peak heart sound point in the peak heart sound point time sequence.

8. A blood pressure measurement apparatus, applied to an electronic blood pressure measurement device, comprising:
a control module configured to control a cuff air bladder to depressurize when the cuff air bladder is in a state of pressurization completion;
a collecting module configured to collect a heart sound signal sequence as well as a cuff pressure time sequence in real-time by a heart sound collecting unit during a depressurization process;
a calculation module configured to calculate a peak heart sound point time sequence based on the heart sound signal sequence; and
a finding module configured to find a cuff pressure value at a corresponding moment in the cuff pressure time sequence as a blood pressure measurement value based on a moment of occurrence of a target peak heart sound point in the peak heart sound point time sequence.

9. An electronic blood pressure measurement device, comprising a memory and a processor, wherein
the processor is configured to execute a computer program stored on the memory; and
the processor, when executing the computer program, realizes the steps of:
controlling a cuff air bladder to depressurize when the cuff air bladder is in a state of pressurization completion;
collecting a heart sound signal sequence as well as a cuff pressure time sequence in real time by a heart sound collecting unit during a depressurization process;
calculating a peak heart sound point time sequence based on the heart sound signal sequence; and
finding a cuff pressure value at a corresponding moment in the cuff pressure time sequence as a blood pressure measurement value based on a moment of occurrence of a target peak heart sound point in the peak heart sound point time sequence.

10. The electronic blood pressure measurement device of claim 9, wherein the processor realizes the step of calculating the peak heart sound point time sequence based on the heart sound signal sequence by:
intercepting a first heart sound signal sequence N seconds before the heart sound signal sequence, and sorting the first heart sound signal sequence in ascending order to obtain a second heart sound signal sequence;
calculating an average value of M heart sound signals after the second heart sound signal sequence to obtain a noise amplitude threshold;
obtaining valid heart sound signals from the heart sound signal sequence based on the noise amplitude threshold to be deposited into a heart sound vector, and recording a time vector corresponding to the heart sound vector; and
calculating the peak heart sound point time sequence based on the heart sound vector and the time vector.

11. The electronic blood pressure measurement device of claim 10, wherein the processor realizes the step of calculating the peak heart sound point time sequence based on the heart sound vector and the time vector by:
performing a difference operation on all the valid heart sound signals in the heart sound vector to obtain a difference sequence;
performing a peak detection on the difference sequence and the time vector based on a predetermined peak detection formula to obtain a peak point sequence and a corresponding time sequence; and
determining the peak heart sound point time sequence based on the peak point sequence and the corresponding time sequence.

12. The electronic blood pressure measurement device of claim 11, wherein the peak detection formula is expressed as:
$${h}_{peaks} = \{ \begin{matrix}{diff}_{peaks}^{i} * {diff}_{peaks}^{i + 1} < 0 & {h}_{time}^{j} = {t}_{peaks}^{i} \\ other & {h}_{time}^{j} = [ ]\end{matrix} ;$$
wherein hₚₑₐₖₛ denotes the peak point sequence, hₜᵢₘₑ denotes the time sequence, diffₚₑₐₖₛ denotes the difference sequence, and tₚₑₐₖₛ denotes the time vector.

13. The electronic blood pressure measurement device of claim 9, wherein the processor realizes the step of controlling the cuff air bladder to depressurize by:
controlling the cuff air bladder to depressurize at a first gas flow rate during a first depressurization phase;
controlling the cuff air bladder to depressurize at a second gas flow rate during a second depressurization phase when a first heart sound signal is captured by the heart sound collecting unit;
controlling the cuff air bladder to depressurize at a third gas flow rate during a third depressurization phase when the heart sound collecting unit a last heart sound signal is collected;
wherein the first gas flow rate is greater than the second gas flow rate, and the third gas flow rate is greater than the first gas flow rate.

14. The electronic blood pressure measurement device of claim 13, wherein the processor realizes the step of controlling the cuff air bladder to depressurize at the second gas flow rate during the second depressurization phase by:
dividing the second depressurization phase into a plurality of sub-depressurization phases and controlling the cuff air bladder to depressurize at a corresponding second gas flow rate selected from a set of preset second gas flow rate values in the plurality of the sub-depressurization phases.

15. The electronic blood pressure measurement device of claim 9, wherein the processor realizes the step of finding the cuff pressure value at the corresponding moment in the cuff pressure time sequence as the blood pressure measurement value based on the moment of occurrence of the target peak heart sound point in the peak heart sound point time sequence by:
finding a cuff pressure value at a corresponding moment in the cuff pressure time sequence as a high-pressure measurement value of the blood pressure based on a moment of occurrence of a first peak heart sound point in the peak heart sound point time sequence, and finding a cuff pressure value of a corresponding moment in the cuff pressure time sequence as a low-pressure measurement value of the blood pressure based on a moment of occurrence of a last peak heart sound point in the peak heart sound point time sequence.
